(19) United States
(12) Patent Application Publication
(10) Pub. No.: US 2025/0228267 A1
(43) Pub. Date: Jul. 17, 2025

(54) ANIMAL FEED OR ANIMAL FEED ADDITIVE COMPRISING CANADINE AND/OR A CANADINE DERIVATIVE

(71) Applicant: PHYTOBIOTICS Futterzusatzstoffe GmbH, Eltville (DE)

(72) Inventor: Hermann ROTH, Eltville (DE)

(73) Assignee: PHYTOBIOTICS Futterzusatzstoffe GmbH, Eltville (DE)

(21) Appl. No.: 18/844,475

(22) PCT Filed: Mar. 10, 2023
(86) PCT No.: PCT/EP2023/056174

§ 371 Date: Sep. 6, 2024

(30) Foreign Application Priority Data
Mar. 11, 2022 (EP) 22161642.8

(51) Int. Cl. A23K 20/137; A23K 10/30; A23K 50/30; A23K 50/75

(52) U.S. Cl. CPC A23K 20/137; A23K 10/30; A23K 50/30; A23K 50/75

(57) ABSTRACT

The invention relates to an animal feed, drinking water or animal feed additive comprising Berberrubine and Canadine and/or a Canadine derivative.

### FIELD OF THE INVENTION

The invention relates to an animal feed additive for optimizing the meat-to-fat ratio in livestock.

### BACKGROUND

Food of animal origin is an important part of the human diet. In the last 20 years, there has been an increased demand for meat with a high muscle-to-fat ratio, as this meat is considered to be particularly healthy. It contains less fat, especially cholesterol, which is considered a health risk, but a higher proportion of protein. The reduction in fat content is most noticeable in pork. Whereas 100 grams of pork fillet contained almost 9 grams of fat in 1991, today it is just 2 grams.

A high proportion of lean meat and a low fat content are used as the basis for various measures of meat quality. For example, so-called “butcher's pigs” must have a lean meat content of at least 60% and a maximum bacon size of 12 to 14 mm. Depending on the meat size, a maximum of 16 to 20 mm of bacon is permitted in order to be classified in commercial class E (55% and more), while 22 to 26 mm of bacon, depending on the meat size, represents the limit from commercial class U (50 to 55%) to class R (45 to 50%). The bacon measure has a very high influence on the classification of the meat in the various commercial and quality classes, as the bacon measure is weighted 4.5 times more heavily than the meat measure in the currently valid formula. If the bacon measure increases by 2 mm, the meat measure must therefore increase by 9 mm in order to avoid a deterioration in the meat classification.

However, the methods used to date to achieve an optimum, i.e. high, meat-to-fat ratio are associated with various disadvantages. They often result in a considerable loss of usable animal material and/or increased production costs.

For example, it is possible to design the meat cuts in such a way that the adjacent fat is cut away from the outset. However, the trimmed fat has a significantly lower market value, so that the costs for the feed used to build it up can be regarded as lost or at least sub-optimally used. Fat that is located within the muscle tissue cannot be removed in this way.

Another way of producing meat with the desired high fat-to-fat ratio is to use breeding breeds with meat optimized for this purpose. However, this can be associated with various disadvantages: existing stables or housing concepts may be unsuitable for said breeding breeds, said breeds may be expensive or susceptible to disease and/or unsuitable for the prevailing climate. Adapting the housing facilities to the needs of these specific breeds may involve considerable costs and reduce the farmer's flexibility to meet changing customer requirements at short notice.

Another option is to slaughter very young animals that have hardly put on any fat. However, this can lead to the animals being slaughtered before the age at which optimum growth (measured by the amount of meat produced per feed quantity) is reached. Production efficiency decreases and costs increase.

The provision of additional exercise can increase the lean meat content, but production costs also increase considerably.

Another approach is to adapt the feed in such a way that a high meat-to-fat ratio and/or the lowest possible fat content is achieved within the limits of what is genetically possible for the breed used. For example, feedstuffs are used that have a particularly high proportion of protein and amino acids, especially lysine. In addition, the energy content of the feed can be reduced during final fattening in order to prevent the animals from becoming fatty. However, there are also limits to this approach, e.g. due to the price of the feed components or additives and the physiological requirements of the respective animal species for the feed.

### Technical Problem and Underlying Solutions

Accordingly, the underlying object of the invention is to provide an alternative or improved animal feed or drinking water or a corresponding animal feed additive.

The problems underlying the invention are respectively solved using the features of the independent patent claims. Embodiments of the invention are specified in the dependent claims. The subsequently explained embodiments and examples are freely combinable with one another, in so far as they are not mutually exclusive.

In one aspect, the invention relates to an animal feed, drinking water or animal feed additive comprising canadine and/or a canadine derivative.

In several tests it was observed that the administration of feed containing canadine or canadine derivative to farm animals such as pigs has a markedly positive effect on the muscle-to-fat ratio and the fat content: The fat content decreases significantly and the muscle content increases. This provides the livestock farmer with a further means of changing the muscle-to-fat ratio in addition to or as an alternative to the methods known in the prior art, or of reducing the fat content in order to improve the quality of the meat. For example, by adding Canadine to the feed during finishing, a livestock farmer could allow the animals to continue to receive a feed with a commonly used energy content and thus reach slaughter weight faster than animals that have received energy-reduced feed during finishing, without reducing the lean meat content in the slaughtered animal. Alternatively, by adding Canadine or a Canadine derivative to an energy-reduced feed during the final fattening period, the livestock farmer could ensure that the animals have a particularly high lean meat content, a particularly low fat content and a particularly high muscle-to-fat ratio at the end of the fattening period. This ratio would be higher than achievable through the administration of energy-reduced feed alone.

Compared to at least some of the methods known in the prior art, the administration of feed or drinking water containing canadine or canadine derivatives also has the particular advantage of greater flexibility: the applicant has found that canadine or derivatives of canadine increase the muscle-to-fat ratio in a large number of animal species, including, for example, cattle, pigs, chickens, and others. In order to achieve a desired high muscle-to-fat ratio, it is therefore not necessary to choose a specific breed of animal for fattening and adapt the husbandry conditions to it. Rather, the livestock farmer can flexibly control and adjust the lean meat content and the fat content of the animals by adding Canadine in a few months, depending on the expected wishes of the buyers.

In addition, the livestock farmer can react flexibly to various unforeseeable situations during fattening. For example, it can happen that towards the end of fattening a significant proportion of the animals in a livestock house become infected with a pathogen that weakens the infected animals. In such a situation, it is not advisable to give energy-reduced feed, as the infected animals need energy to fight the pathogen. However, feed is dispensed centrally in large stables. If the animals not infected by the pathogen are given high-calorie feed, there is a risk that the healthy animals will become obese, i.e. have an undesirably high fat content. In such a situation, the livestock owner must therefore weigh up the risk of further worsening the health of the already infected animals and increasing the risk of the pathogen spreading to other animals by administering energy-reduced feed for final fattening, against the risk of helping the infected animals by administering high-energy feed, but impairing the fattening quality of the non-infected animals. In such situations, the administration of feed or drinking water containing Canadine or a Canadine derivative, optionally in combination with medication to treat an animal disease (such as coccidiosis, diarrhea, Newcastle disease, etc.) can ensure that high meat quality is also guaranteed in situations in which a high-energy feed must be administered due to the presence or suspected presence of a disease. Also in other situations in which it is to be feared that the meat quality will be too low with regard to the fat and lean meat content of the slaughter animals, e.g. due to the sex, breed of the animals, age, state of health or due to the feed used, Canadin-containing feed or drinking water can ensure that there is a significant shift in the muscle-to-fat ratio in favor of the muscle content and to the detriment of the fat content.

Canadine, also known as tetrahydroberberine or under the CAS number 522-97-4, is a benzylisoquinoline alkaloid (BIA) from the structural subgroup of protoberberine alkaloids. Like the other protoberberine alkaloids, canadine comprises a benzylisoquinoline structural unit. Canadine therefore belongs to the group of alkaloids.

A canadine derivative is understood here to be a physiologically compatible substance which is derived from canadine as the parent compound, i.e. which contains the basic structure of canadine, but also one or more functional groups. In particular, the canadine derivative may be a methylated form of canadine with an identical or similar effect on the meat and fat content of an animal as canadine. For example, the canadine derivative may be single or multiple methylated variants of canadine. In particular, the canadine derivative may be single-methylated canadine, especially N-methylcanadine. For example, the N-methylcanadine may be the (S)—N-methylcanadine with the CHEBI number 16512.

According to one embodiment, the animal feed or drinking water has a concentration of at least 0.16 μg of canadine and/or canadine derivative per kg of animal feed or drinking water, preferably at least 1.6 μg of canadine and/or canadine derivative per kg of animal feed or drinking water, for example from 1 μg to 50 μg of canadine and/or canadine derivative per kg of animal feed or drinking water.

According to another embodiment, it is an animal feed additive which, when added as intended to an animal feed or to a drinking water, produces an animal feed or drinking water with a concentration of at least 0.16 μg of canadine and/or canadine derivative per kg of animal feed or drinking water, preferably from 0.16 μg to 50 μg of canadine and/or canadine derivative per kg of animal feed or drinking water. Preferably at least 1.6 μg of canadine and/or canadine derivative per kg of animal feed or drinking water, e.g. from 1 μg to 50 μg of canadine and/or canadine derivative per kg of animal feed or drinking water.

For example, the animal feed additive may be a powder or granules comprising canadine and/or a canadine derivative, optionally one or more further alkaloids and typically other substances, e.g. fillers, minerals, vitamins and/or flavorings. The animal feed additive may also comprise plant material, e.g. crushed plant parts such as leaves or stems and/or plant extracts, in which the alkaloids are typically contained. The fillers facilitate the dosing and homogeneous addition of the animal feed additive to a significantly larger amount of animal feed or water, so that the final concentration of the active ingredients can be adapted to the needs of the animals and the respective application situation. The animal feed additive can also be in liquid form. The latter is particularly advantageous if the additive is to be mixed with water to provide a Canadin-containing drinking water of a specific concentration. For example, the canadine and/or canadine derivative can be kept in solution by lowering the pH of the liquid feed additive (e.g. by means of organic acids such as citric acid).

According to one embodiment, the animal feed, the animal feed additive or the drinking water comprises berberrubine.

This embodiment is based on the surprising observation that the use of a combination of canadine and/or a canadine derivative in combination with berberrubine may be particularly beneficial, as berberrubine appears to enhance the effect of the canadine or canadine derivative. By administering a combination of both substances, a significantly greater improvement in the lean meat-to-fat ratio was achieved than was observed when canadine was administered alone. The shelf life of the meat also improved.

For example, the berberrubine may be comprised in the animal feed, animal feed additive or drinking water in the form of plant material. For example, the plant material may be material from the plants Thalictrum aquilegifolium, Berberis heteropoda, material from branches of Berberis actinacantha or from the stems of Berberis darwinii or Berberis valdiviana. Berberis vulgaris, Thalictrum polygamum, Fibraurea chloroleuca (Berberidaceae, Ranunculaceae, Menispermaceae), Macleya cordata and other plants may also comprise berberrubine in sufficient concentration or be obtained in the form of an extract. For example, the extract may be an aqueous ethanolic extract of plant material.

According to one embodiment, the ratio of the canadine component (canadine and/or canadine derivative) to berberrubine in the animal feed, animal feed additive and/or drinking water is in the range from 1:5 to 5:1, e.g. in the range from 1:1.2 to 1.2:1.

According to embodiments of the invention, the animal feed, animal feed additive or drinking water comprises dried parts of the plant Maclaeaya cordata, e.g. leaves and/or roots, flowers, capsules and/or parts of the stem. The flower and the capsule in particular, but also the root, comprise canadine and in particular a high proportion of N-methyl-canadine. By combining leaves with different proportions of flowers, capsules and/or roots, the desired concentration of canadine and N-methyl-canadine can be easily adjusted depending on the intended use and active ingredient content of the plant. The dried parts are preferably ground into a powder beforehand to simplify dosing and mixing.

According to one embodiment, the animal feed or drinking water comprises at least 1 μg of berberrubine per kg of animal feed or drinking water, in particular at least 10 μg of berberrubine per kg of animal feed or drinking water. However, the berberrubine concentration can also be higher, e.g. in the range of 1-400 μg berberrubine per kg animal feed or drinking water.

According to a further embodiment, the animal feed additive has a berberrubine concentration such that, when added as intended to an animal feed or to a drinking water, an animal feed or drinking water with at least 1 μg of berberrubine per kg of animal feed or drinking water, in particular at least 10 μg of berberrubine per kg of animal feed or drinking water, is obtained. Concentrations of 1-400 μg of berberrubine per kg of animal feed or drinking water are also possible.

According to some embodiments, the animal feed or the drinking water or the animal feed additive used for their production may comprise further alkaloids such as sanguinarine and/or chelerythrine, which may have various positive effects, e.g. an improvement in feed conversion.

According to one embodiment, the animal feed, the animal feed additive or the drinking water is intended for administration to farm animals, pets or hobby animals. The animals may in particular be poultry, pigs, cattle, sheep, goats, horses, and/or rabbits.

According to some embodiments, the canadine comprised in the animal feed, drinking water or animal feed additive is synthetically or biotechnologically, in particular microbiotechnologically produced canadine.

For example, canadine can be obtained in several steps from a disubstituted phenylethylamine and a disubstituted benzaldehyde, as described, for example, in Li W et al, “Total Synthesis of (−)-Canadine, (−)-Rotundine, (−)-Sinactine, and (−)-Xylopinine Using a Last-Step Enantioselective Ir-Catalyzed Hydrogenation”, Journal Organical Chemistry 2021 Jun. 18; 86(12):8143-8153. doi: 10.1021/acs.joc.1c00602. Epub 2021 Jun. 2. PMID: 34076443. The use of synthetic canadine has the advantage of facilitating exact dosing. When using plant material, additional steps may be required to concentrate, purify and/or determine the concentration of canadine in the plant material used.

According to other embodiments, the canadine is present as a component of plant material, in particular a plant extract, in the animal feed, drinking water or animal feed additive.

For example, the animal feed, the animal feed additive or the drinking water may comprise plant material of Canadian orange root or a plant of the Papaveraceae family, such as Corydalis yanhusuo and Corydalis turtschaninovii. According to another example, the plant material may come in whole or in part from the five-seeded plume-poppy (Macleaya cordata), in particular from the aerial part, i.e. stems and/or leaves.

According to embodiments, the animal feed or drinking water comprises at least 10 mg of Macleaya cordata leaves per kg of animal feed or drinking water, in particular at least 100 mg of Macleaya cordata leaves per kg of animal feed or drinking water. Preferably, the animal feed or drinking water is free of other plant organs of Macleaya cordata (such as roots, stems, flowers, etc.) or comprises these other plant organs in an amount of less than 20%, in particular less than 10%, e.g. less than 5% or less than 1% of the amount of Macleaya cordata leaves per kg of animal feed or drinking water. The composition of the animal feed additive and the amount of Macleaya cordata leaves in the additive is such that, when dosed as intended, an appropriate animal feed or drinking water is obtained. This can be advantageous because M. cordata leaves comprise a comparatively high concentration of N-methyl-canadine in particular and the leaves make up a large part of the biomass of M. cordata plants and are easy to process (e.g. pulverize) and homogeneously distributable.

According to some embodiments, the canadine derivative comprised in the animal feed, drinking water or animal feed additive is a synthetically, biotechnologically and/or microbiotechnologically produced canadine derivative. For example, a production process for N-methyl-canadine is described in European patent application EP3221461A1.

According to embodiments of the invention, the animal feed, drinking water or animal feed additive comprises a mixture of canadine and the canadine derivative.

According to embodiments of the invention, the animal feed, drinking water or animal feed additive comprises a mixture of synthetic and/or biotechnologically produced canadine and/or canadine of plant origin. Additionally or alternatively, the animal feed, drinking water or animal feed additive may be a mixture of the synthetic and/or biotechnologically produced canadine derivative and/or the canadine derivative of plant origin. For example, a mixture of synthetic and plant-based active ingredient can be used to specifically set a desired active ingredient concentration so that natural fluctuations in the active ingredient content of plants can be compensated for. However, it is also possible, for example, to obtain a desired active ingredient concentration on a purely plant basis by selecting the appropriate proportion of different plant organs (leaves, stems, roots, flowers, etc.).

According to some embodiments, the canadine, the canadine derivative and/or the berberrubine is present in the animal feed, drinking water or animal feed additive as a physiologically compatible salt, in particular as chloride or sulfate.

Administration in the form of salts can have the advantage that the alkaloid is stabilized. It is assumed that this is because the salt anion prevents the accumulation of hydroxyl ions, which can inactivate the alkaloid.

According to some embodiments, the animal feed, drinking water or animal feed additive is one for reducing the fat content in the animal body and/or carcass.

According to some embodiments, the animal feed, drinking water or animal feed additive is one for increasing the muscle-to-fat ratio in the meat of animals. According to some embodiments, the animal feed, drinking water or animal feed additive is one for use as a medicament, e.g. an animal feed, drinking water or animal feed additive for treating obesity muscle weakness or pathologically increased oxidative stress.

According to some embodiments, the animal feed, drinking water or animal feed additive is one for increasing the shelf life of the meat of slaughtered animals.

According to some embodiments, the animal feed, drinking water or animal feed additive is one for the preventive or acute treatment of oxidative stress in the tissue of animals, in particular during fattening.

In a further aspect, the invention relates to an animal feed, drinking water or animal feed additive comprising berberrubine. According to some embodiments, the animal feed additionally comprises canadine and/or a canadine derivative. According to embodiments, the dosage, concentration, uses and other properties described herein for the canadine-containing animal feed, drinking water or animal feed additive also apply to the animal feed, drinking water or animal feed additive comprising berberrubine (without canadine and without canadine derivative or optionally supplemented with canadine and/or the canadine derivative).

In a further aspect, the invention relates to a use of an animal feed, drinking water or animal feed additive according to one of the embodiments described herein for reducing the fat content in the animal body and/or in the carcass of the animals.

In a further aspect, the invention relates to a use of an animal feed, drinking water or animal feed additive according to one of the embodiments described herein for increasing the muscle-to-fat ratio in the meat of animals and/or in the carcass of the animals.

In a further aspect, the invention relates to a use of an animal feed, drinking water or animal feed additive according to one of the embodiments described herein for reducing the fat content in the meat of animals and/or in the carcass of animals.

In a further aspect, the invention relates to a use of an animal feed, drinking water or animal feed additive according to one of the embodiments described herein for increasing the shelf life of the meat of slaughtered animals.

In a further aspect, the invention relates to a use of an animal feed, drinking water or animal feed additive according to one of the embodiments described herein for the preventive or acute treatment of oxidative stress in the tissues of animals, in particular during fattening. In particular, said animal feed, drinking water or animal feed additive can be used for the preventive or acute treatment of pathological, i.e. medically treatable, oxidative stress in the tissue of animals.

Oxidative stress is a metabolic condition of an organism in which an excess of reactive oxygen species (ROS) leads to damage to the affected cells. These reactive oxygen species are produced as part of metabolic processes such as the respiratory chain. Examples of such compounds are the superoxide anion radical O2-, hydrogen peroxide (H2O2) and the hydroxyl radical —OH. Normally, a cell is able to render a certain amount of reactive oxygen compounds harmless through neutralization. Reducing substances are produced and stored for this purpose. Accordingly, oxidative stress is such an imbalance between oxidizing and reducing substances that this normal repair and detoxification function of the cell is overwhelmed and consequently all cellular and extracellular macromolecules can be damaged.

The consequences of high oxidative stress include lipid peroxidation, protein oxidation and DNA damage. These three processes are considered to be partly responsible for the ageing process and a deteriorated state of health. Some forms of severe oxidative stress are to be regarded as pathological physiological conditions requiring veterinary treatment, as they restrict the growth and performance of animals in a similarly significant way as is the case with many well-known animal diseases such as bird flu, calf diarrhea or coccidiosis.

High-grade, especially pathological oxidative stress occurs when the cell metabolism is significantly out of balance. This can be caused, for example, by a diet low in vital substances, poor husbandry conditions, infections, inflammation and other health problems.

In some embodiments, the animal feed, drinking water or animal feed additive is used to improve various meat parameters such as fat content, fat to lean meat ratio, shelf life, etc. In some embodiments, the animal feed, drinking water or animal feed additive is used as a medicament, e.g. to treat obesity, or muscle weakness or a morbidly poor general condition due to a high level of oxidative stress in the tissue. These problems can be signs of ageing or caused by a poor or unbalanced diet. Medicinal uses are particularly beneficial for pets and hobby animals, but can also be used successfully in livestock farming, e.g. for fattening pigs, cattle and poultry.

In a further aspect, the invention relates to a container which comprises the animal feed additive and which additionally comprises an information carrier, e.g. a print or sticker, or is provided together with the information carrier (e.g. leaflet). It is also possible that the information carrier comprises a reference (URL, e.g. a URL encoded in a OR code) to a dosing instruction, which is provided in electronic form, e.g. as a website. A dosage is specified on the information carrier (and/or on the referenced electronic information carrier), i.e. a specification of that amount of animal feed additive which, when used as intended, must be added to a certain amount of animal feed or drinking water in order to obtain an animal feed or drinking water with a concentration of canadine and/or canadine derivative and/or berberrubine as described herein for embodiments of the invention. The container may be, for example, a bucket, can, bag, sack or significantly smaller or larger containers, depending on whether the amount is intended for medicinal use to treat individual pets or for regular feed addition by larger fattening operations.

In a further aspect, the invention relates to a use of an animal feed, drinking water or animal feed additive according to any of the embodiments described herein for medicinal purposes in animals. For example, the animal feed, drinking water or animal feed additive can be used for the treatment of obesity in pets requiring medical treatment, pathologically high concentrations of free oxygen radicals (oxidative stress) in the tissue or pathologically low formation of muscles or muscle protein.

In a further aspect, the invention relates to a use of an animal feed additive according to one of the embodiments described herein for the preparation of an animal feed or drinking water for the preventive or acute treatment of oxidative stress in the tissues of animals.

In a further aspect, the invention relates to a use of an animal feed additive according to one of the embodiments described herein for the preparation of an animal feed or drinking water for the reduction of the fat content in the animal body.

In a further aspect, the invention relates to a use of an animal feed additive according to one of the embodiments described herein for the preparation of an animal feed or drinking water for increasing the muscle-to-fat ratio and/or for reducing the fat content in the meat of animals.

According to embodiments, the use of the animal feed additive, animal feed or drinking water is characterized in that, when the animal feed additive, the animal feed and/or the drinking water is dosed as intended, at least 0.016 μg of canadine and/or canadine derivative per kg of live weight per day, e.g. at least 0.16 μg of canadine and/or canadine derivative per kg of live weight. e.g. of at least 0.16 μg of canadine and/or canadine derivative per kg of live weight, e.g. of at least 1 μg of canadine and/or canadine derivative per kg of live weight is administered. Typically, the amount of canadine and/or canadine derivative administered per day does not exceed 4.8 μg canadine and/or canadine derivative per kg live weight. The comparatively high concentration of canadine and/or canadine derivative of around 5 μg per kg live weight can be used in particular for the treatment of pathological physiological conditions or a greatly increased level of oxidative stress. An even higher dosage is harmless. For example, it has been observed that canadine and/or canadine derivative can be administered for the treatment of particularly high oxidative stress in an amount of over 5 μg, e.g. of over 1 mg, e.g. also over 50 mg canadine and/or canadine derivative per day and kg live weight of the animal, without undesirable side effects occurring.

It has been observed that said low levels of canadine and/or canadine derivative are suitable to improve the muscle/fat ratio and to reduce the level of oxidative stress in the tissues of the animals (which is relevant for the storability and quality of the meat).

If said feed, drinking water or feed additive is used for the treatment of pathological metabolic conditions, e.g. for the treatment of pathological oxidative stress, the concentration of canadine and/or canadine derivative per kg live weight and day can also be chosen to be significantly higher, e.g. such that the concentration is at least twice or at least five times or at least 10 times higher than the above-mentioned minimum concentrations.

According to embodiments, the animal feed or drinking water comprises at least 0.16 μg of canadine and/or canadine derivative per kg of animal feed or drinking water, in particular with at least 1.6 μg of canadine and/or canadine derivative per kg of animal feed or drinking water. Typically, the animal feed or drinking water comprises 0.1 to 50 μg of canadine and/or canadine derivative per kg of animal feed or drinking water. The concentration of canadine in the animal feed additive is preferably chosen such that the animal feed additive, when used as intended and added to a feed or drinking water, results in an animal feed or drinking water with the above-mentioned canadine and/or canadine derivative concentrations.

According to embodiments, the animal feed or drinking water additionally comprises berberrubine.

According to some embodiments, the animal feed or drinking water comprises at least 0.01 μg of berberrubine per kg of animal feed or drinking water, in particular at least 0.1 μg of berberrubine per kg of animal feed or drinking water.

According to other embodiments, it is an animal feed additive whose berberrubine concentration is selected such that, when added as intended to an animal feed or to a drinking water, an animal feed or drinking water with at least 0.01 μg of berberrubine per kg of animal feed or drinking water, in particular with at least 0.1 μg of berberrubine per kg of animal feed or drinking water, is obtained.

The term “plant material” is understood here to mean material which is wholly or partly of plant origin. For example, the plant material may be parts of plants, e.g. pulverized leaves or stems. It may also be a liquid material, e.g. a plant extract, in particular an alcohol-based plant extract, e.g. an ethanol-based plant extract

### BRIEF DESCRIPTION OF THE DRAWINGS

In the following, embodiments of the invention are explained in more detail by way of example only, reference being made to the drawings in which they are comprised, in which:

FIG. 1 shows a table with results of a feeding trial with pigs;

FIG. 2 shows a bar chart illustrating the result of the feeding trial according to FIG. 1;

FIG. 3 shows a table showing the results of another feeding trial with broilers;

FIG. 4 shows a bar chart illustrating the results of the table in FIG. 3;

FIG. 5 shows a bar chart illustrating the effect of different concentrations of canadine on the protein and fat content of chicken breast meat;

FIG. 6 shows a bar chart showing the effect of different amounts of canadine on malondialdehyde (MDA) and oxidative stress;

FIG. 7 shows the effect of canadine on the breast and leg muscles of the test animals;

FIG. 8 shows the structural formula of Canadin; and

FIG. 9 shows the structural formula of (S)—N-methylcanadine.

### DETAILED DESCRIPTION

The following descriptions relate to various tests in which a feed composition according to the invention was used. The corresponding advantages and efficacies also apply analogously to a drinking water with a corresponding alkaloid quantity or concentration, as well as to an animal feed additive for the production of said animal feed.

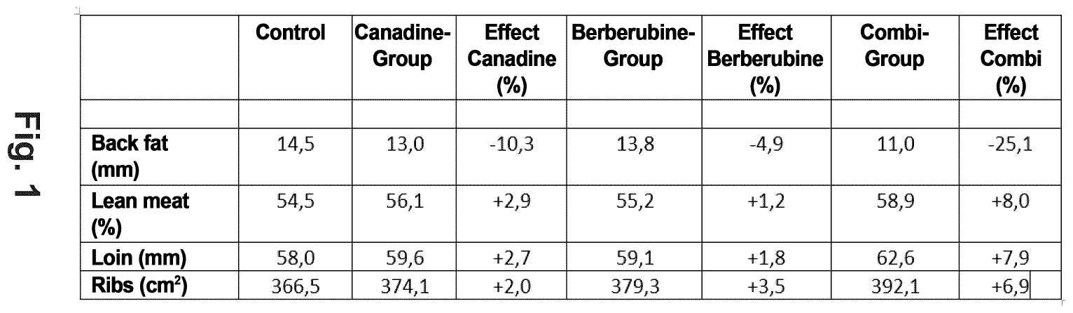

FIG. 1 shows a table with the results of a feeding test.

995 fattening pigs, comprising both male and female animals, were divided into four groups of approximately equal size. A control group, referred to as the “control”, was fed a fattening diet without added canadine (and without added alkaloids) for 30 days until the day of slaughter (wheat-based basal diet). From the 30th day before slaughter, the group designated as the “Canadine group” was fed a fattening diet with the same composition as the control group, but which additionally comprised N-methyl-canadine at a concentration of 35 μg N-methyl-canadine per kg of feed. The Canadine group's feed did not comprise berberrubine. From the 30th day before slaughter, the group designated as the “berberrubine” group received a fattening diet with the same composition as the control group's diet, but which also comprised berberrubine at a concentration of 35 μg berberrubine per kg of feed. Canadine was not comprised in the diet of the Canadine group. The group referred to as the “combi” group received a fattening diet during the 30 days prior to slaughter that had the same composition as the diet of the control group, but which additionally comprised a combination of N-methyl-Canadine and berberrubine at a concentration of 35 μg N-methyl-Canadine per kg feed and 35 μg berberrubine per kg feed.

The animals were fed ad libitum with the respective feed of the group until the day of slaughter, so they could consume as much food as they wanted. After slaughter, various parts of the carcass were measured and the results recorded in the table.

As a result, the use of a feed containing N-methyl-canadine led to a higher carcass weight (110 kg-112 kg). The thickness of the back fat was significantly reduced (14.5 mm-13 mm, −10.3%). The muscle-to-fat ratio increased significantly due to the increased muscle growth and the reduction in fat content. Muscle growth was observed in the loin (+2.7%) and the meat attachment to the ribs increased significantly (+2%). Muscle thickness, measured in mm, increased slightly. Overall, more muscle mass (protein) was produced with a reduced fat content.

The aforementioned positive effects were also observed in the berberrubine group.

It was observed that the positive effects of N-methyl-Canadine were synergistically enhanced to a considerable extent by the additional administration of berberrubine. For example, backfat thickness decreased approximately twice as much (−25.1%) when a combination of N-methyl-Canadine and berberrubine was fed compared to a diet containing only N-methyl-Canadine.

The control group served as the basis for calculating the difference percentage values in each case

In the test shown here, N-methyl-Canadine and berberrubine were administered in a 1:1 ratio. Further tests have shown that other ratios also significantly enhance the effect of canadine or N-methyl-canadine, e.g. a ratio of canadine (or derivative):berberrubine of 1:1.2 to 1.2:1, or of 1:5 to 5:1.

The two alkaloids were each present in the feed in the form of salts, namely as canadine chloride and berberrubine chloride.

The test shows that feeding N-methyl-canadine to fattening pigs approx. 1 month before slaughter significantly reduces backfat density and also increases the lean meat content. Body areas such as loin or ribs, which already have a high lean meat content from the outset, grow significantly better under the effect of N-methyl-Canadine than without the addition of Canadin. An animal feed containing N-methyl-canadine therefore leads to an improvement, i.e. an increase, in the muscle-to-fat ratio in pigs. This positive effect is considerably enhanced by the additional administration of berberrubine.

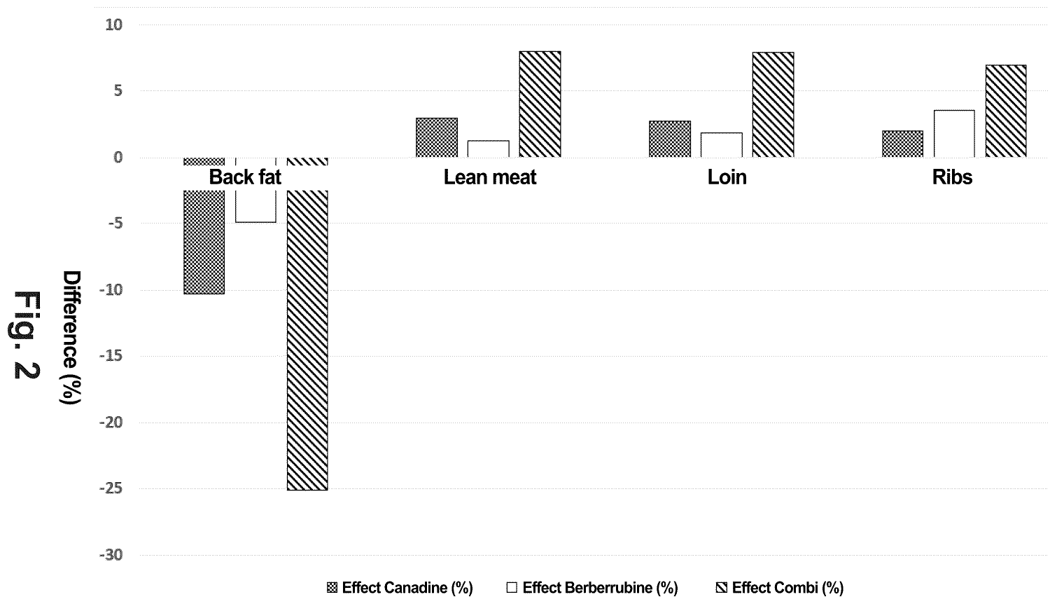

FIG. 2 shows the results of the feeding test described with regard to FIG. 1 in the form of a bar chart, which shows the relative changes to the control group (“difference”) in percentage values. The synergistic effect of the combined administration of N-methyl-canadine and berberruibine to reduce the fat content while simultaneously increasing the muscle content can be clearly seen. For example, N-methyl-canadine alone caused a 10.3% reduction in backfat, berberrubine alone caused a 4.9% reduction, but a combination of both substances caused a reduction of over 25%. A synergistic effect was also observed with regard to the increase in lean meat content. It can also be seen from the diagram that N-methyl-canadine and berberrubine alone already have corresponding positive effects.

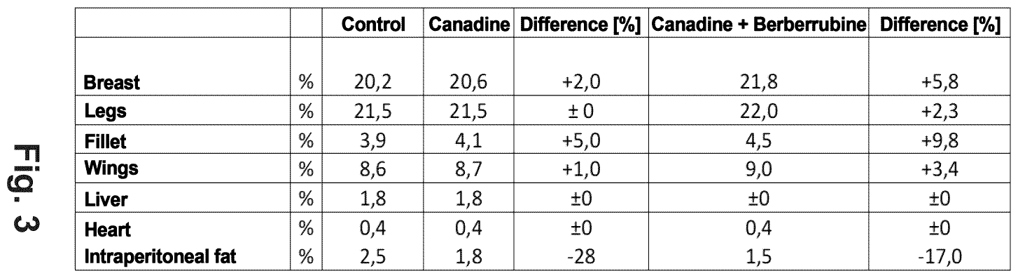

FIG. 3 shows a table illustrating the results of another feeding test with chickens. 26,400 Cobb 500 chickens were divided into three equal groups (“control”, “Canadine” and “Candin+Berberrubine”). All three groups were fed ad libitum for 49 days. Feeding was carried out in three phases (“starter” day 1 to 10, “grower” day 11-24, “finisher” 25-49) according to the changing feed composition requirements during the development of the chickens. One-day-old chicks were used. The diet was based on corn, soybean meal, wheat, pureed, and the respective alkaloid additive.

The diet of the Canadine group comprised an additional 35 μg canadine per kg feed, but no berberrubine. The feed of the Canadine+berrubine group comprised 35 μg canadine per kg feed and an additional 35 μg berberrubine per kg feed.

The chickens were then slaughtered and various organs and carcass parts were examined more closely with regard to fat and muscle content.

The Canadine group achieved better slaughter results, particularly in the breast, wing and fillet areas; the intraperitoneal fat content was significantly lower in the Canadine group and in the Canadine+berrubine group than in the control group. The fat content was 2.5% in the control group, 1.8% in the Canadine group and only 1.5% in the Canadine+berrubine group. This corresponds to a difference in fat content of 28% between the Canadine group and the control group. Thus, a significant improvement in the meat-to-fat ratio was observed in both chickens and pigs. In the Canadine+Berberrubine group, an additional 17% reduction in fat percentage was achieved compared to the Canadine group.

Overall, as in pigs, it can be concluded that Canadine or N-methyl-Canadine leads to a higher lean meat content with reduced fat content, i.e. a higher lean meat content, and that this effect is enhanced by berberrubine.

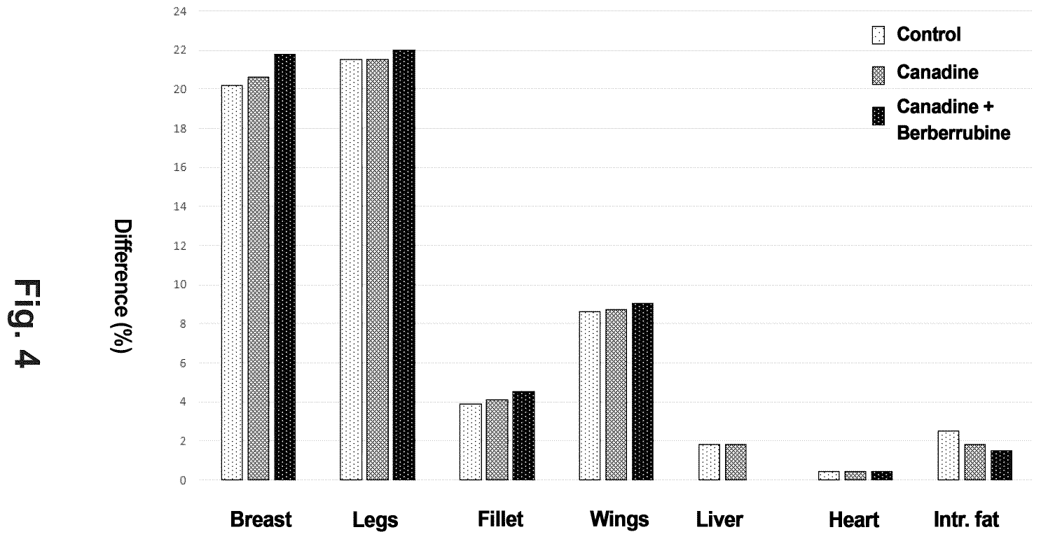

FIG. 4 shows a bar chart illustrating the results of the test with the broilers shown in FIG. 3.

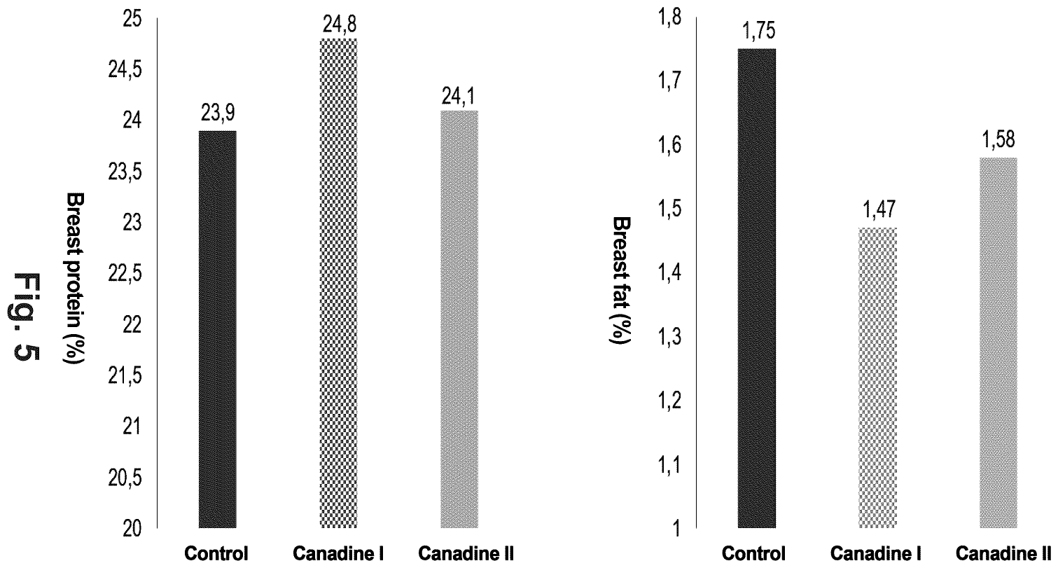

FIG. 5 shows the results of a further test with broilers in the form of a bar chart.

360 one-day-old chicks were used. The diet was based on corn, soybean meal, wheat, pureed, and the additive. The trial comprised three growth and feeding phases: “starter” (day 1 to 10), “grower” (day 11-24) and “finisher” (day 25-42); three testal groups—control, two concentrations, 120 animals per testal batch. Feeding (ad libitum) was carried out with a basal diet, which was supplemented depending on the group.

A control group designated as “control” was fed a basic diet adapted to the respective growth phase without the addition of canadine (and without the addition of other alkaloids). The basic feed of all groups consisted mainly of maize, soybean meal and wheat. Depending on the group, the feed may also comprise the appropriate amount of alkaloids. The group referred to as the “Canadine I” group was fed a basic diet that had the same composition as the diet of the control group, but which also comprised canadine at a concentration of 50 μg per kg of feed. Berberrubine was not comprised in the feed of the three groups. The group designated as the “Canadine II” group received a basic diet that had the same composition as the diet of the control group, but comprised canadine at a concentration of 35 μg per kg of feed, i.e. less than in the “Canadine I” group.

The animals were fed ad libitum with the respective feed of the group until the day of slaughter, so they could eat as much as they wanted. After slaughter, various measurements were carried out on the animals' carcasses.

For example, the protein and fat content of the breast fillet of the broilers was determined. The Canadine group showed a significantly reduced fat content and a significantly increased muscle content compared to the control group.

The same effect was observed in the Canadine II group, but it was less pronounced than in the “Canadine I” group. It could thus be shown that the beneficial effect of canadine increases with its concentration.

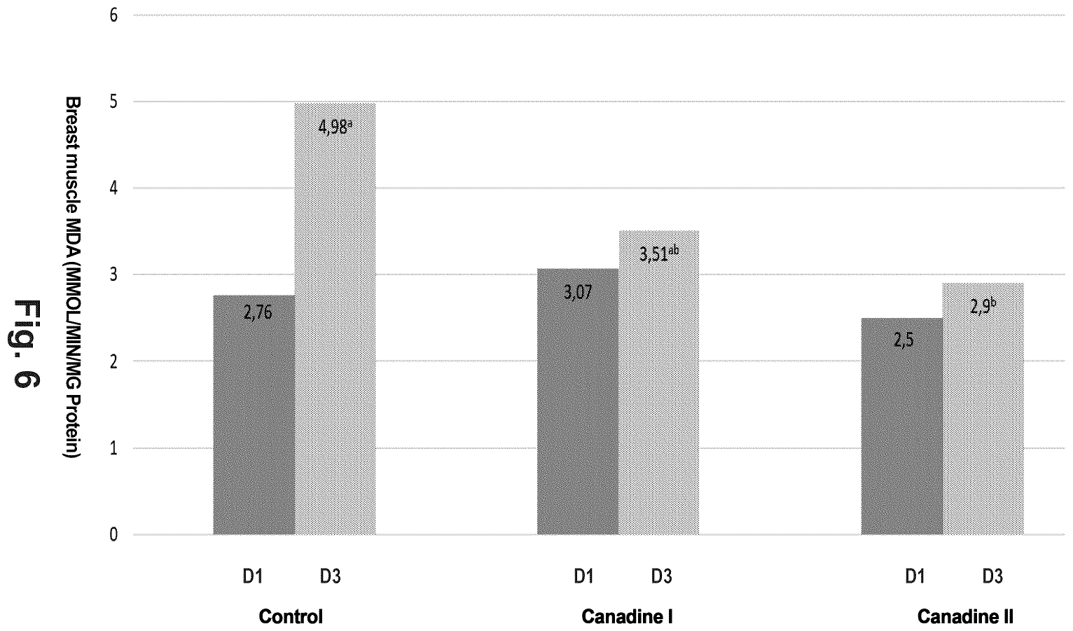

FIG. 6 shows that canadine can be used to treat oxidative stress. This can lead, for example, to an improvement in shelf life.

To determine the oxidative stress in a tissue, the content of malondialdehyde (MDA) in this tissue is generally determined. MDA is therefore a biomarker for the level of oxidative stress. Elevated MDA levels in tissue can be an expression of unfavorable living conditions during the lifetime of an organism, e.g. incorrect feed, diseases or unfavorable husbandry conditions. After slaughter, however, the MDA value can also serve as an indicator of a decline in meat quality during storage. During storage, the MDA content of the tissue increases due to the penetration of atmospheric oxygen into the tissue and due to biochemical reactions that continue to take place in the tissue. A high MDA content is therefore an indicator that the meat has been stored for a long time.

In the breast muscle of the broilers from the feeding test described in FIG. 4, the MDA value was determined after slaughter. In each of the three groups, the MDA value was determined in half of the animals after one day of storage in the refrigerator (subgroup “D1”) and in the other half of the animals after three days of storage in the refrigerator (subgroup “D3”).

In the control group, the MDA content increased significantly during storage in the refrigerator. This is an indication of free oxygen radicals in the breast muscles of the control group and an indication of a decrease in storability or ageing of the carcass. However, this increase was significantly lower in the breast fillets of the broilers in the “Canadine I” and “Canadine II” groups up to the third day (“D3”). This shows that the administration of Canadine was able to significantly reduce the amount of reactive oxygen compounds in the breast muscles of the broilers and thus the storability of the carcass. It should be noted that the MDA concentration at slaughter is subject to a certain variability in the different animals, so that the relative increase in the MDA concentration measured on the third day D3 relative to the value measured on the first day of storage (D1) or on the day of slaughter is particularly relevant for the storability. While the MDA value of the control thus increases by over 80% from D1 to D3, the MDA value of the two Canadine groups only increases by around 16%.

Reducing oxidative stress by administering a feed according to embodiments of the invention thus not only promotes animal health, but also leads to a significant improvement in storability.

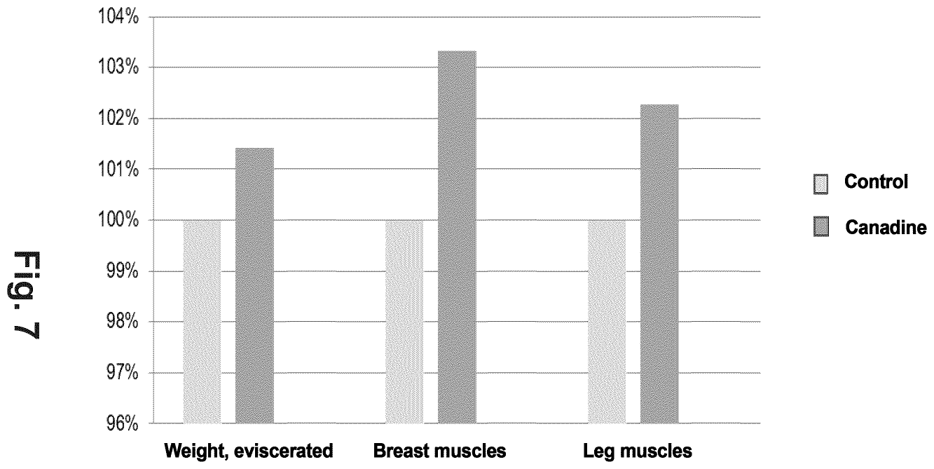

FIG. 7 shows the results of a further test with 300 broilers, which were divided into two groups of approximately equal size. The control group received a basic feed without any other physiologically active additives. The Canadine group received the basic feed comprising an additional 35 μg of Canadine per kg of feed. Feeding was carried out according to this scheme during the weaning period of the broilers (43-49 days after birth) (ad libitum).

Subsequently, all animals were slaughtered and various parameters were determined. The weight of the carcass was measured, excluding the innards, and the weight of the breast and leg muscles. The carcass yield and the weight of the breast muscles were significantly increased in the Canadine group compared to the control group.

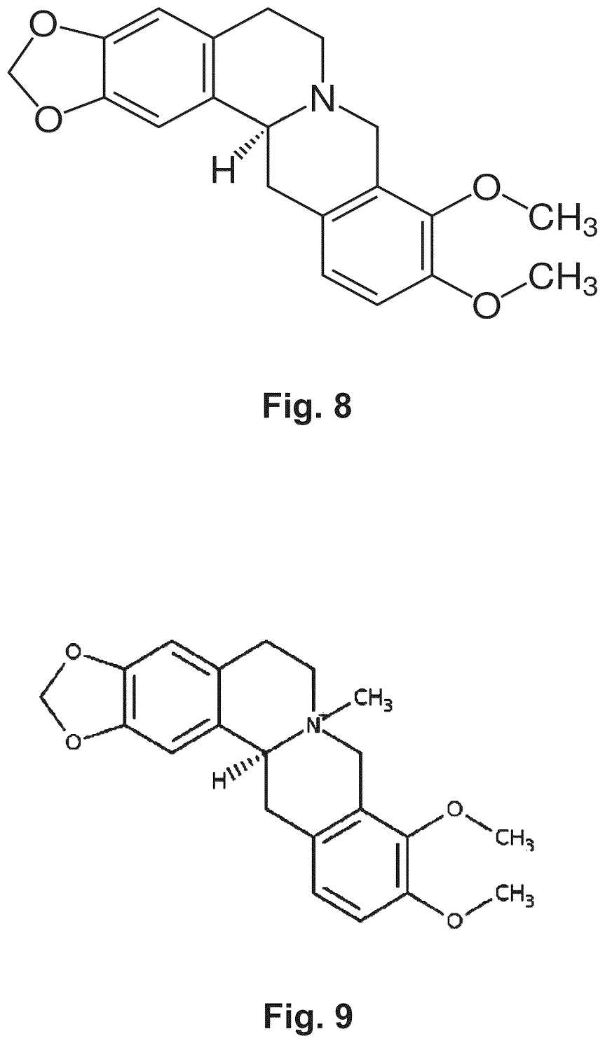

FIGS. 8 and 9 show the structural formulae of canadine and (S)—N-methylcanadine, respectively. It was found that both substances have the desired effects, in particular reducing the fat content in the carcass and increasing the proportion of lean meat.

Further, supplementary tests WV1, WV2 and WV3 were carried out on the efficacy of canadine alone and in combination with berberrubine. The active ingredients methyl canadine and berberrubine used in all three tests were obtained by chromatographic purification of an extract of five-seeded plume-poppy.

In test WV1, 600 pigs were divided into 7 groups of approximately equal size. All groups received the same basic feed throughout the last month before slaughter, but the groups differed with regard to the amount of canadine (methyl-canadine) or berberrubine administered. Gruppe G K: Kontrollgruppe, erhielt weder Canadine noch Berberrubin
Group GCA: 5 μg Canadine per kg feed (no berberrubine)
Group GCB: 10 μg Canadine per kg feed (no berberrubine)
Group GBA: 20 μg berberrubine per kg feed (no canadine)
Group GBB: 40 μg berberrubine per kg feed (no canadine)
Group GCBA: 5 μg canadine and 20 μg berberrubine per kg feed
Group GCBB: 10 μg canadine and 40 μg berberrubine per kg feed

After slaughter, the thickness of the rump fat, loin, ribs and the lean meat percentage of the total meat was determined. The results of the test are as follows:

| | | | | | | GCBA | GCBB |
|---|---|---|---|---|---|---|---|
| | | GCA | GCB | GBA | GBB | Canadine/ | Canadine/ |
| | GK | Canadine | Canadine | Berberrubine | Berberrubine | Berberrubine: | Berberrubine |
| Tissue | Control | 5 μg | 10 μg | 20 μg | 40 μg | 5 μg/20 μg | 10 μg/40 μg |
| backfat [mm] | 14.00 | 12.60 | 12.80 | 12.70 | 12.60 | 11.2 | 11.00 |
| lean meat [%] | 53.80 | 55.40 | 56.05 | 55.05 | 55.32 | 57.73 | 58.20 |
| loin [mm] | 56.00 | 57.60 | 57.82 | 57.95 | 58.25 | 59.98 | 59.92 |
| ribs [cm2] | 364.5 | 371.80 | 372.20 | 373.02 | 373.87 | 383.10 | 384.32 |

This corresponds to a percentage increase or decrease relative to the control group of:

| | | | | | | GCBA | GCBB |
|---|---|---|---|---|---|---|---|
| | | GCA | GCB | GBA | GBB | Canadine/ | Canadine/ |
| | GK | Canadine | Canadine | Berberrubine | Berberrubine | Berberrubine: | Berberrubine |
| | Control | 5 μg | 10 μg | 20 μg | 40 μg | 5 μg/20 μg | 10 μg/40 μg |
| Tissue | (K) | %* | %* | %* | %* | %* | %* |
| Backfat [mm] | 100 | −10 | −8.57 | −9.28 | −10 | −20 | −21.43 |
| Lean meat [%] | 100 | +2.9 | +4.18 | +2.3 | +2.82 | +7.30 | +10.82 |
| Loin [mm] | 100 | +2.85 | +3.25 | +3.48 | +4.02 | +7.10 | +7.00 |
| Ribs [cm2] | 100 | +2.0 | +2.11 | +2.34 | +2.57 | +5.10 | 5.44 |

A synergistic, over-additive effect of the two substances was also observed here: for example, a reduction in backfat of −8.57% was observed in the GOB group, −10% in the GBB group and −21.43% in the GCBB group. The lean meat content increased significantly more in the GCBA and GCBB groups than was to be expected based on the effects of individual substances observed in the GCA and GBA or GOB and GBB groups.

In a further test WV2, 600 broiler chicks were divided into 7 groups of approximately equal size. All groups received the same basic feed (pelletized complete feed) during the entire start, growth and end phase of fattening, but the groups differed with regard to the amount of canadine (methyl-canadine) or berberrubine administered.
Group GK: control group, received neither canadine nor berberrubine
Group GCA: 25 μg canadine per kg feed (no berberrubine)
GCB group: 50 μg canadine per kg feed (no berberrubine)
Group GBA: 50 μg berberrubine per kg feed (no canadine)
Group GBB: 100 μg berberrubine per kg feed (no canadine)
Group GCBA: 25 μg canadine and 50 μg berberrubine per kg feed
GCBB group: 50 μg canadine and 100 μg berberrubine per kg feed

After slaughter, the weight of different parts of the carcass (as a percentage of the total weight) was determined. The results of the experiment are as follows:

| | | GCA | GCB | GBA | GBB | GCBA |
|---|---|---|---|---|---|---|
| | GK | Canadine | Canadine | Berberrubine | Berberrubine | Canadine/Berberrubine: |
| Tissue | Control | 25 μg | 50 μg | 50 μg | 100 μg | 25 μg/50 μg |
| Breast | 20.7 | 21.3 | 21.5 | 21. 6 | 21.8 | 22.3 |
| [%] | | | | | | |
| Leg | 21.1 | 21.2 | 21.6 | 21.9 | 21.7 | 22.4 |
| [%] | | | | | | |
| Fillet | 4.0 | 4.2 | 4.3 | 4.5 | 4.6 | 4.7 |
| [%] | | | | | | |
| Wings | 8.4 | 8.9 | 9.1 | 9.15 | 9.2 | 9.4 |
| [%] | | | | | | |
| Liver | 1.7 | 1.8 | 1.8 | 1.8 | 1.8 | 1.8 |
| [%] | | | | | | |
| Heart | 0.5 | 0.5 | 0.5 | 0.55 | 0.5 | 0.5 |
| [%] | | | | | | |
| Fat | 2.3 | −2.0 | −2.0 | −2.0 | −1.9 | −1.8 |
| (i.p.) | | | | | | |
| [%] | | | | | | |

Values in relation to the control group (=100%)

| | | | | | | GCBA | GCBB |
|---|---|---|---|---|---|---|---|
| | | GCA | GCB | GBA | GBB | Canadine/ | Canadine/ |
| | GK | Canadine | Canadine | Berberrubine | Berberrubine | Berberrubine: | Berberrubine |
| Tissue | Control | 25 μg | 50 μg | 50 μg | 100 μg | 25 μg/50 μg | 50 μg/100 μg |
| Breast [%] | 100 | +2.90 | +3.86 | +4.34 | +5.31 | +7.7 | +8.21 |
| Leg [%] | 100 | ±0 | +2.37 | +3.79 | +2.84 | +6.16 | +6.64 |
| Fillet [%] | 100 | +5.00 | +7.50 | +11.25 | +11.50 | +17.50 | +25.00 |
| Wings [%] | 100 | +5.96 | +8.33 | +8.93 | +9.52 | +11.90 | +13.10 |
| Liver [%] | 100 | +5.88 | +5.88 | +5.88 | +5.88 | +5.88 | +5.88 |
| Heart [%] | 100 | ±0 | ±0 | ±0 | ±0 | ±0 | ±0 |
| Fat (i.p.) [%] | 100 | −13.04 | −13.04 | −13.10 | −17.39 | −21.74 | −39.13 |

Here also a clear effect on the muscle content (corresponding to the proportion of breast and fillet in particular) as well as on the fat content was observed. The proportion of breast meat and fillet was significantly increased, while the fat content decreased significantly when Canadine and/or berberrubine were administered. In the GCBB group, the fat content decreased by over 39% relative to the control group.

In a further test WV3, 500 broiler chickens were divided into 7 groups of approximately equal size and given group-specific feed in accordance with the experimental description for experiment WV2. After slaughter, the weight of different parts of the carcass (as a percentage of the total weight) was determined. The results of the experiment are as follows:

| | | | | | | GCBA | GCBB |
|---|---|---|---|---|---|---|---|
| | | GCA | GCB | GBA | GBB | Canadine/ | Canadine/ |
| | GK | Canadine | Canadine | Berberrubine | Berberrubine | Berberrubine: | Berberrubine |
| | Control | 25 μg | 50 μg | 50 μg | 100 μg | 25 μg/50 μg | 50 μg/100 μg |
| Protein content in | 23.9 | 24.8 | 24.5 | 25.0 | 24.3 | 26.5 | 27.0 |
| breast meat [%] | | | | | | | |
| Fat content in | 1.75 | 1.66 | 1.62 | 1.65 | 1.63 | 1.60 | 1.58 |
| breast meat [%] | | | | | | | |

| | | | | | | GCBA | GCBB |
|---|---|---|---|---|---|---|---|
| | | GCA | GCB | GBA | GBB | Canadine/ | Canadine/ |
| | GK | Canadine | Canadine | Berberrubine | Berberrubine | Berberrubine: | Berberrubine |
| | Control | 25 μg | 50 μg | 50 μg | 100 μg | 25 μg/50 μg | 50 μg/100 μg |
| Deviation of protein | 100 | +3.77 | +2.51 | +4.60 | +1.67 | 10.88 | +12.97 |
| content in breast | | | | | | | |
| meat from GK [%] | | | | | | | |
| Deviation of fat | 100 | −5.14 | −7.43 | −5.71 | −6.86 | −8.57 | 9.71 |
| content in breast | | | | | | | |
| meat from GK [%] | | | | | | | |

For each of the two substances, a clear (positive) effect on the lean meat percentage and a clear (negative) effect on the fat percentage were observed. A combination of the two substances led to an over-additive effect on the promotion of the muscle percentage or reduction of the protein percentage, particularly when higher concentrations were used.

Drinking water and feed have therefore proven to be particularly advantageous with the following examples of substance quantity combinations (each in μg per kg feed or drinking water):

| Canadine only | Berberrubine only | Canadin + Berberrubin combined |
|---|---|---|
| 5 | 10 | 5 + 20 |
| 10 | 20 | 25 + 50 |
| 20 | 40 | 50 + 100 |
| 25 | 50 | 50 + 100 |
| 40 | 100 | |
| 50 | | |

According to some examples, the feed or drinking water may thus comprise at least 5 μg canadine/kg feed or drinking water, in particular 5-100 μg canadine/kg feed or drinking water.

According to some examples, the feed or drinking water may thus comprise at least 10 μg berberrubine/kg feed or drinking water, in particular 10-150 μg berberrubine/kg feed or drinking water.

According to some examples, the feed or drinking water may comprise a combination of at least 5 μg canadine and at least 10 μg berberrubine/kg feed or drinking water. For example, the feed or drinking water may comprise a combination of at least 1 μg canadine and at least 1 μg berberrubine/kg feed or drinking water. In particular, the feed or drinking water may comprise a combination of 5-100 μg canadine and 10-150 μg berberrubine/kg feed or drinking water.

A feed additive is also disclosed herein which, when dosed as intended, produces one of the animal feeds or drinking waters described herein.

1. Animal feed, drinking water or animal feed additive comprising berberrubine and canadine and/or a canadine derivative,
wherein it is an animal feed or drinking water with a concentration of at least 0.01 μg of berberrubine per kg of animal feed or drinking water and with at least 0.16 μg of canadine and/or canadine derivative per kg of animal feed or drinking water, or
wherein it is an animal feed additive which, when added to an animal feed or to a drinking water as intended, results in an animal feed or drinking water with a concentration of at least 0.01 μg of berberrubine per kg of animal feed or drinking water and with at least 0.16 μg of canadine and/or canadine derivative per kg of animal feed or drinking water.

2. Animal feed, drinking water or animal feed additive according to claim 1,
wherein it is animal feed or drinking water with a concentration of at least 1.6 μg of canadine and/or canadine derivative per kg of animal feed or drinking water, or
wherein it is an animal feed additive which, when added to an animal feed or drinking water as intended, results in an animal feed or drinking water with a concentration of at least 1.6 μg of canadine and/or canadine derivative per kg of animal feed or drinking water

3. Animal feed, drinking water or animal feed additive according to claim 1,
wherein it is an animal feed or drinking water with at least 1 μg of berberrubine per kg of animal feed or drinking water, in particular with at least 10 μg of berberrubine per kg of animal feed or drinking water; or
wherein it is an animal feed additive which, when added to an animal feed or to a drinking water as intended, produces an animal feed or drinking water with at least 1 μg of berberrubine per kg of animal feed or drinking water, in particular with at least 10 μg of berberrubine per kg of animal feed or drinking water.

4. Animal feed or drinking water according to claim 1, wherein said feed or drinking water comprises a combination of at least 1 μg, in particular at least 5 μg, of canadine and at least 1 μg, in particular at least 10 μg, of berberrubine/kg of feed or drinking water, or animal feed additive according to any of the preceding claims, wherein said animal feed additive, when dosed as intended, results in an animal feed or drinking water with the said amount of canadine and berberrubine.

5. Animal feed, drinking water or animal feed additive according to claim 1, wherein the canadine derivative is an at least monomethylated canadine, in particular N-methyl-canadine.

6. Animal feed, drinking water or animal feed additive according to claim 1 for administration to farm animals, domestic animals or hobby animals, in particular poultry, pigs, cattle, sheep, goats, horses, or rabbits.

7. Animal feed, drinking water or animal feed additive according to claim 1,
wherein canadine is synthetic or biotechnologically produced canadine; and/or
wherein the canadine derivative is a synthetic or biotechnologically produced canadine derivative; and/or
wherein the canadine is present as a component of plant material in the animal feed, drinking water or animal feed additive; and/or
wherein the canadine derivative is present as a constituent of plant material in the animal feed, drinking water or animal feed additive.

8. Animal feed, drinking water or animal feed additive according to claim 1, wherein the canadine and/or the canadine derivative is present as a physiologically compatible salt, in particular as chloride or sulphate.

9. Animal feed, drinking water or animal feed additive according to claim 1 for use as a medicament.

10. Animal feed, drinking water or animal feed additive according to claim 1 for reducing the fat content in the animal body.

11. Animal feed, drinking water or animal feed additive according to claim 1 for increasing the muscle-to-fat ratio in the meat of animals or use of an animal feed, drinking water or animal feed additive according to claim 1 for increasing the muscle-to-fat ratio in the meat of animals.

12. Animal feed, drinking water or animal feed additive according to claim 1 for the preventive or acute treatment of oxidative stress in the tissues of animals, or use of an animal feed, drinking water or animal feed additive according claim 1 for the preventive or acute treatment of oxidative stress in the tissues of animals.

13. Animal feed, drinking water or animal feed additive according to claim 1 for increasing the storability of the meat of slaughtered animals or use of an animal feed, drinking water or animal feed additive according to claim 1 for increasing the storability of the meat of slaughtered animals.

14. Use of an animal feed, drinking water or animal feed additive according to claim 1 for reducing the fat content in the animals.

15. Use of an animal feed, drinking water or animal feed additive according to claim 1 for medical purposes.

16. Use of an animal feed additive according to claim 10, wherein the use of the animal feed additive comprises an admixture to an animal feed or to a drinking water in such an amount that an animal feed or drinking water is produced with a concentration of at least 0.16 μg of canadine and/or canadine derivative per kg of animal feed or drinking water, in particular of at least 1.6 μg of canadine and/or canadine derivative per kg of animal feed or drinking water.
